(19) United States
(12) Patent Application Publication
(10) Pub. No.: US 2026/0216471 A1
(43) Pub. Date: Jul. 30, 2026

(54) FLEXIBLE ELECTRICAL COMPONENTS AND MEDICAL DEVICES INCORPORATING THE SAME

(71) Applicant: St. Jude Medical, Cardiology Division, Inc., St. Paul, MN (US)

(72) Inventors: Troy Tegg, Elk River, MN (US); Hong Cao, Maple Grove, MN (US)

(21) Appl. No.: 19/629,686

(22) Filed: Mar. 26, 2026

Related U.S. Application Data
(63) Continuation-in-part of application No. 19/274,224, filed on Jul. 18, 2025.
Application No. 19/274,224 is a continuation-in-part of application No. PCT/US2024/059776, filed on Dec. 12, 2024.
(60) Provisional application No. 63/609,625, filed on Dec. 13, 2023.
(60) Provisional application No. 63/575,077, filed on Apr. 5, 2024.
(60) Provisional application No. 63/702,909, filed on Oct. 3, 2024.

(51) Int. Cl. A61M 25/00; A61L 29/14

(52) U.S. Cl. CPC A61M 25/0012; A61L 29/14; A61M 25/0043; A61M 2025/0058

(57) ABSTRACT

A flexible electrode includes a flexible, electrically insulative substrate, a bonding layer disposed on a first surface of the substrate, a conductive layer disposed on a second surface of the substrate, and a via through the bonding layer and the substrate that exposes a portion of the reverse surface of the conductive layer. The exposed portion of the reverse surface can be secured (e.g., soldered) to a conductive contact, such as a conductive pill, on an exterior surface of a catheter shaft. The flexible electrode can then be wrapped around the exterior surface of the shaft to form a ring electrode and the bonding layer can be bonded to the catheter shaft, such as by reflow bonding.

### CROSS-REFERENCE TO RELATED APPLICATIONS

This application is a continuation-in-part of U.S. application Ser. No. 19/274,224, filed 18 Jul. 2025 (“the '224 application”), now pending, which is a continuation-in-part of international patent application no. PCT/US24/59776, filed 12 Dec. 2024 (“the '776 PCT”), which claims the benefit of U.S. provisional Application No. 63/609,625, filed 13 Dec. 2023 (“the '625 provisional”), U.S. provisional Application No. 63/575,077, filed 5 Apr. 2024 (“the '077 provisional”), and U.S. provisional Application No. 63/702,909, filed 3 Oct. 2024 (“the '909 provisional”). The '224 application, '776 PCT, '625 provisional, '077 provisional, and '909 provisional are hereby incorporated by reference in their entireties as though fully set forth herein.

### FIELD

The present disclosure relates generally to elongate medical devices, such as catheters. In particular, the instant disclosure relates to elongate medical devices carrying various sensors, for example in their distal segments.

### BACKGROUND

Catheters are used for an ever-growing number of procedures. For example, catheters are used for diagnostic, therapeutic, and ablative procedures, to name just a few examples. In an electrophysiology (“EP”) procedure, for example, a catheter may be manipulated through the patient's vasculature and to an intended site for mapping and/or treatment, for example, a site within the patient's heart.

A catheter may carry one or more devices, sensors, or surgical instruments, such as electrodes, which may be used for ablation, diagnosis, and/or the like. In many extant catheters, these sensors are embedded into the catheter shaft by swaging.

Swaging has certain disadvantages, however. For instance, the sensors must be relatively uniform metal bands in order to be swaged onto the catheter shaft. This makes swaging unsuitable for non-metallic and/or asymmetric sensors.

### BRIEF SUMMARY

Disclosed herein is a flexible electrode including: a flexible, electrically insulative substrate; a bonding layer disposed on a first surface of the substrate; a conductive layer disposed on a second surface of the substrate opposite the first surface of the substrate, the conductive layer having an exposed surface and a reverse surface, wherein the reverse surface is adjacent the second surface of the substrate; and a via through the bonding layer and the substrate that exposes a portion of the reverse surface of the conductive layer.

The flexible electrode may have a rectangular plan shape that, when wrapped about a cylindrical core, defines a ring electrode. It is also contemplated that the rectangular plan shape of the flexible electrode can include a flared portion proximate the via.

The substrate can include polyimide. The bonding layer can include a melt-processable material, such as one or more of polyether block amide (PEBA) and polyurethane.

The conductive layer can include one or more of platinum, iridium, copper, gold, nickel, and palladium.

Also disclosed herein is a method of manufacturing a catheter. The method includes: forming a catheter shaft including a conductive contact adjacent an exterior surface thereof; forming a flexible electrode including: a flexible, electrically insulative substrate; a bonding layer disposed on a first surface of the substrate; a conductive layer disposed on a second surface of the substrate opposite the first surface of the substrate, the conductive layer having an exposed surface and a reverse surface, wherein the reverse surface is adjacent the second surface of the substrate; and a via through the bonding layer and the substrate that exposes a portion of the reverse surface of the conductive layer; securing the portion of the reverse surface of the conductive layer to the conductive contact; wrapping the flexible electrode about the exterior surface of the catheter shaft to form a ring electrode; and bonding the bonding layer to the catheter shaft.

The step of securing the portion of the reverse surface of the conductive layer to the conductive contact can include soldering the portion of the reverse surface of the conductive layer to the conductive contact.

The step of bonding the bonding layer to the catheter shaft can include reflow bonding the bonding layer to the catheter shaft. A heat shrink can be placed around the flexible electrode prior to reflow bonding the bonding layer to the catheter shaft.

The conductive contact may include a conductive pill adjacent the exterior surface of the catheter shaft. Alternatively, the conductive contact includes a first segment of an elongate electrical conductor, wherein a second segment of the elongate electrical conductor extends through the catheter shaft.

The foregoing method of manufacture offers various advantages over extant methods. For instance, because it bonds without swaging, it permits the use of non-circular (e.g., partial ring and/or ring segment) and non-metallic sensors. As another advantage, the reflow bonding process offers additional sealing against fluid ingress around electrodes. It also reduces cost and complexity of manufacture.

The instant disclosure also provides a catheter including: an elongate shaft; and a ring electrode mounted to the elongate shaft. The ring electrode includes: a flexible, electrically insulative substrate; a bonding layer disposed on a first surface of the substrate; a conductive layer disposed on a second surface of the substrate opposite the first surface of the substrate, the conductive layer having an exposed surface and a reverse surface, wherein the reverse surface is adjacent the second surface of the substrate; and a via through the bonding layer and the substrate that exposes a portion of the reverse surface of the conductive layer.

The elongate shaft can also include a conductive contact, wherein the exposed portion of the reverse surface is bonded to the conductive contact. The exposed portion of the reverse surface can be soldered to the conductive contact. An elongate electrical conductor can extend through the elongate shaft from the conductive contact. Further, the conductive contact can include a segment of the elongate electrical connector.

It is contemplated that the bonding layer can be reflow bonded to the elongate shaft.

The ring electrode can also include a flared portion proximate the via.

The catheter may be used for crossing the interatrial septum using radiofrequency energy.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a side, partially cut-away view illustration of the Seldinger technique for vascular access.

FIG. 2 is a cross-sectional, close-up view of region 2 in FIG. 1.

FIG. 3 is a perspective view of an exemplary introducer according to embodiments of the instant disclosure.

FIG. 4 is a close-up view of the distal region of the exemplary introducer shown in FIG. 3.

FIGS. 5A and 5B are perspective views of a flexible ring electrode according to aspects of the instant disclosure.

FIG. 6 is a close-up view of region 6 in FIG. 5A.

FIG. 7 illustrates the flexible ring electrode of FIGS. 5A and 5B in plan view.

FIG. 8A illustrates the distal region of the introducer of FIG. 3 and, in particular, the conductive contact for a flexible ring electrode.

FIG. 8B illustrates the distal region of the introducer of FIG. 3 with a flexible ring electrode mounted thereon.

FIG. 9A illustrates the distal region of the introducer of FIG. 3; the outermost portions of the shaft have been removed to reveal aspects of the construction that would otherwise be obscured, such as a braided reinforcing layer and a spiral-wound flexible circuit including conductive pills for a plurality of flexible ring electrodes.

FIG. 9B illustrates the flexible circuit of FIG. 9A in plan view prior to being spiral-wound around the introducer of FIG. 3.

FIG. 9C is a close-up of region C in FIG. 9B.

FIG. 9D depicts an exemplary connector at the proximal end of a flexible circuit according to embodiments of the instant disclosure.

FIG. 10 is a close-up illustration of a portion of a transverse cross-section of an introducer shaft having a spirally-wound flexible circuit in accordance with certain embodiments disclosed herein.

FIG. 11A depicts an alternative construction of the distal region of the introducer of FIG. 3 including undulating conductive traces on a flexible circuit as disclosed herein.

FIG. 11B depicts a flexible circuit with undulating conductive traces as disclosed herein.

FIG. 11C is a close-up of region 11C in FIG. 11B.

FIG. 11D is an exploded view of the flexible circuit depicted in FIG. 11B.

FIG. 11E depicts a flexible circuit with interrupted undulating conductive traces.

FIG. 12A is transverse cross-section of an introducer shaft according to aspects of the instant disclosure.

FIG. 12B is a close-up of region 12B in FIG. 12A.

FIG. 13 illustrates the handle of the introducer of FIG. 3, with portions thereof removed to reveal interior features, and a signal cable for an electroanatomical mapping system.

FIG. 14 illustrates the handle of the introducer of FIG. 3 connected to the signal cable of FIG. 13.

FIG. 15 is a schematic diagram of an exemplary anatomical mapping system.

FIGS. 16A-16C are, respectively, front, top, and side views of a flat magnetic sensor such as may be employed in various aspects of the instant disclosure.

FIG. 16D illustrates installation of the flat magnetic sensor of FIGS. 16A-16C into a shaft, such as an introducer shaft.

FIGS. 17A-17C are, respectively, front, top, and side view of a hollow magnetic sensor such as may be employed in various aspects of the instant disclosure.

FIG. 17D illustrates installation of the hollow magnetic sensor of FIGS. 17A-17C into a shaft, such as an introducer shaft.

FIG. 18 illustrates a coronary sinus catheter including a flex circuit having a plurality of zig-zag conductive traces.

FIG. 19A depicts a radiofrequency transseptal crossing device according to aspects of the disclosure.

FIG. 19B is a close-up and cut-away view of the distal tip of the radiofrequency transseptal crossing device shown in FIG. 19A.

FIG. 20A is a cut-away view of proximal electrical connections for the radiofrequency transseptal crossing device shown in FIG. 19A.

FIG. 20B is a close-up view of the electrical connector shown in FIG. 20A.

While multiple embodiments are disclosed, still other embodiments of the present disclosure will become apparent to those skilled in the art from the following detailed description, which shows and describes illustrative embodiments. Accordingly, the drawings and detailed description are to be regarded as illustrative in nature and not restrictive.

### DETAILED DESCRIPTION

Aspects of the instant disclosure relate to mounting various sensors on elongate medical devices. For purposes of illustration, embodiments of the disclosure will be described in connection with mounting a flexible ring electrode on a catheter and, more specifically, a steerable introducer catheter (sometimes also referred to as a “sheath” or “introducer sheath”).

Additional aspects of the instant disclosure relate to elongate medical devices that include flexible electrical components (e.g., flexible electrodes and/or flexible electronic circuits). For purposes of illustration, embodiments of the disclosure will be described in connection with a steerable introducer catheter that may include one or more flexible ring electrodes and/or one or more flexible electronic circuits. It is contemplated, however, that the described features and methods may be incorporated into any number of catheters or similar medical devices, including, but not limited to, steerable diagnostic and therapeutic catheters (e.g., electrophysiology mapping and/or ablation catheters), coronary sinus catheters, fixed curve catheters and introducers, transseptal dilators, intracardiac echocardiography (ICE) catheters, radiofrequency (RF)-based transseptal puncture apparatus, and the like.

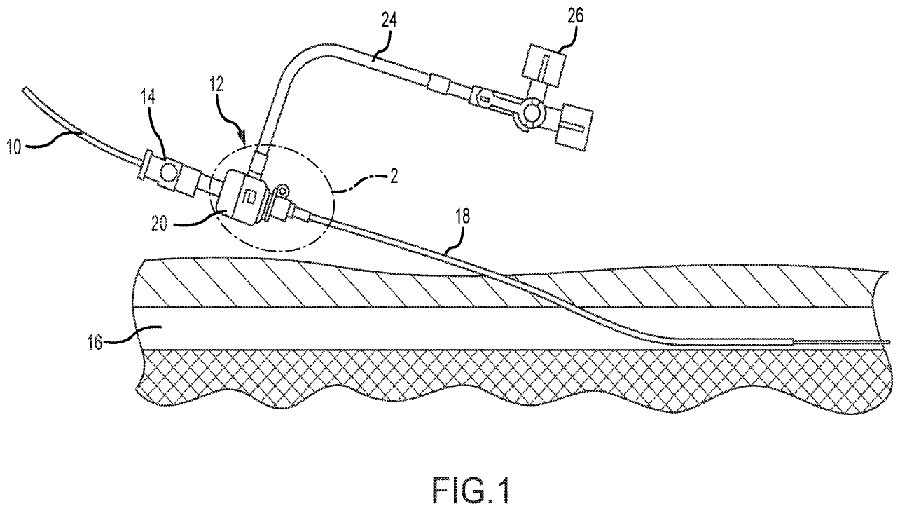

Referring now to the drawings, FIG. 1 depicts the introduction of various medical devices, including guidewire 10, introducer 12, and dilator 14, into a blood vessel 16 using the Seldinger technique. Insofar as the ordinarily-skilled artisan will be familiar with the Seldinger technique, it need not be described in further detail herein. Likewise, those of ordinary skill in the art will be familiar with other approaches to introducing medical devices into blood vessel 16, including the use of steerable introducers.

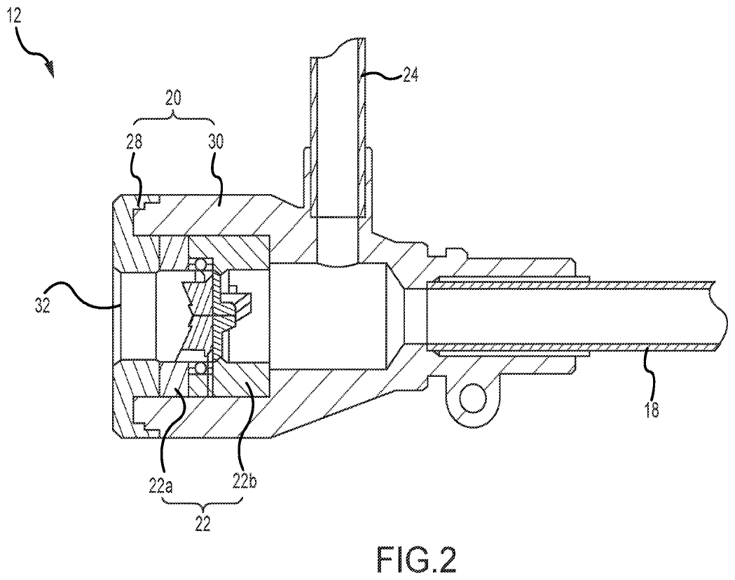

As FIG. 1 illustrates, introducer 12 includes a shaft 18, a hub 20 (which, as discussed further below and as shown in FIG. 2, incorporates a hemostasis valve system 22), and a side-port fluid tubing 24 with an associated stopcock assembly 26. As shown in FIG. 2, hub 20 includes a cap 28 and a housing 30 circumferentially sealed together, within which an integral hemostasis valve system 22 (including, by way of example only, two hemostasis valve gaskets 22a, 22b) is disposed at its proximal end. Side-port fluid tubing 24 and associated stopcock assembly 26 are also coupled to hub 20 or housing 30 to enable the introduction of medical fluids (e.g., saline) through introducer 12 for an intended clinical procedure. Various details of hub 20, including housing 30 and cap 28, will be familiar to those of ordinary skill in the art; thus, hub 20 will only be described herein to the extent necessary to understand the instant disclosure.

The exterior of hub 20 is defined by housing 30 and cap 28. Cap 28 defines an aperture 32 into housing 30 (e.g., an opening through which various medical devices may be inserted through hub 20, into shaft 18, and thus into blood vessel 16.

Contained within circumferentially-sealed housing 30 by cap 28 are one or more hemostasis valve gaskets, such as a first (or proximal) valve gasket 22a and a second (or distal) valve gasket 22b as described in international patent application publication No. WO 2022/245598, which is hereby incorporated by reference as though fully set forth herein. Of course, other hemostasis valve gasket configurations and arrangements are regarded as within the spirit and scope of the present disclosure, and the foregoing reference is merely exemplary rather than limiting. As fully assembled and constrained within housing 30, first and second valve gaskets 22a, 22b may be collectively referred to as a hemostasis valve system 22.

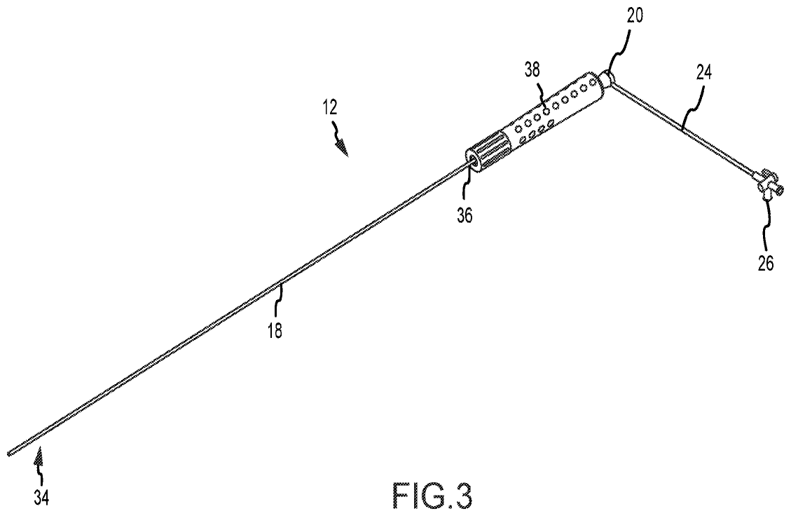

FIG. 3 illustrates additional aspects of introducer 12. As shown in FIG. 3, shaft 18 has a distal region 34 and a proximal end 36. A handle 38 may be coupled to proximal end 36 of shaft 18 to control introducer 12 (e.g., to push, torque, deflect, and/or steer introducer 12). Of course, it is also contemplated that any known device for manipulation of introducer 12 may be coupled to proximal end 36 of shaft 18, including, without limitation, robotic manipulation devices and the like.

Various additional (and, in some instances, optional) aspects of the construction of introducer 12 will be familiar to those of ordinary skill in the art. For example, the ordinarily skilled artisan will appreciate that introducer 12 can be irrigated, such that it can also be coupled to a suitable supply of irrigation fluid and/or an irrigation pump (e.g., a peristaltic pump). As a further example, those of ordinary skill in the art will appreciate that introducer 12 can be equipped with force feedback capabilities (e.g., via the incorporation of one or more force sensors in distal region 34). As yet another example, those of ordinary skill in the art will be familiar with the use of braided and/or helically-wound reinforcing layers embedded within the wall of shaft 18. Insofar as such features are not necessary to an understanding of the instant disclosure, they are neither illustrated in the drawings nor explained in detail herein.

Introducer 12 can also be made steerable, for example by incorporating one or more actuators into handle 38 that are coupled to one or more steering or pull wires that extend through shaft 18 and that terminate in one or more pull rings within distal region 34. The pull wires may make one or more revolutions about the circumference of shaft 18 as they extend along the length thereof as disclosed, for example, in U.S. Pat. No. 11,484,690 , which is hereby incorporated by reference as though fully set forth herein.

FIG. 4 is a close-up of distal region 34 of introducer 12 and illustrates ring electrodes 40 mounted thereon. Although FIG. 4 illustrates two ring electrodes 40, it should be understood that the number and arrangement of electrodes 40 is merely illustrative and that distal region 34 can include any number of electrodes 40, that electrodes 40 may be of various physical configurations (e.g., ring electrodes, segmented ring electrodes, partial ring electrodes) and/or materials (e.g., metallic and/or non-metallic electrodes), that the positioning and/or spacing of electrodes 40 within distal region 34 may vary, and so forth. Those of ordinary skill in the art will appreciate that electrodes 40 may be applied to various diagnostic and/or therapeutic objectives, including visualization and/or localization of distal region 34 of introducer 12 using an electroanatomical mapping system, electrophysiological sensing and/or mapping, and the like.

Moreover, distal segment 34 may include non-electrode diagnostic and/or therapeutic elements, such as positioning sensors (e.g., magnetic coil localization sensors), pressure sensors, force sensors, and the like. Thus, the term “sensors” is used herein to refer not only to electrodes 40, but also to other diagnostic and/or therapeutic elements that may be mounted within distal region 34 and/or elsewhere along shaft 18.

Electrodes 40 may be conventional ring electrodes that may be swaged and/or laser welded onto shaft 18. Those of ordinary skill in the art will be familiar with such electrodes and techniques, such that further explanation is not required herein.

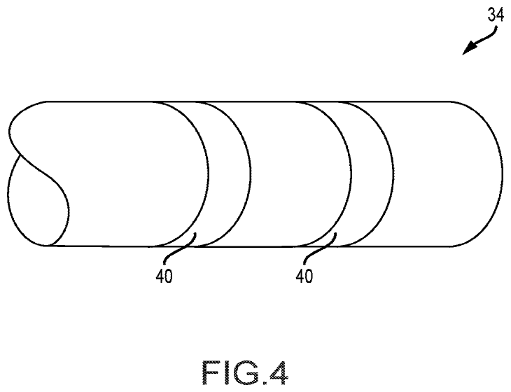

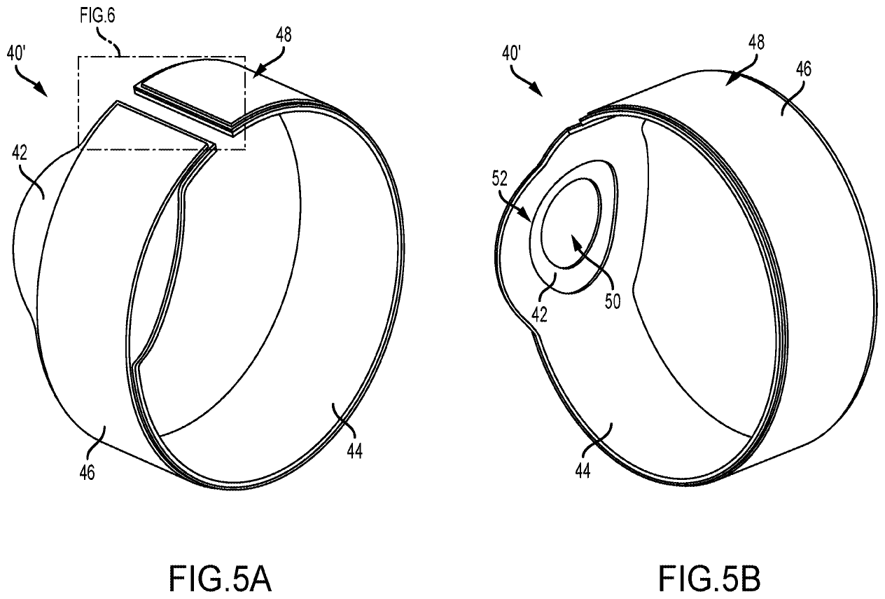

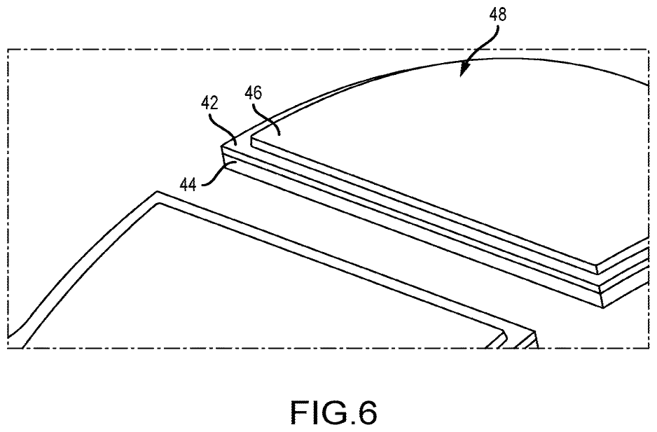

Alternatively, electrodes 40 may be flexible ring electrodes 40′, the construction of which can be understood with reference to FIGS. 5A, 5B, and 6. As shown in FIGS. 5A, 5B, and 6, electrodes 40 (e.g., flexible ring electrodes 40′) generally have a three-layer construction, including a flexible substrate 42, a bonding layer 44, and a conductive layer 46.

Substrate 42 is typically made of an electrically insulative material. One suitable material for substrate 42 is polyimide, though other electrically insulative materials are regarded as within the scope of the present disclosure. Substrate 42 provides strength and dimensional stability to electrode 40 (e.g., flexible ring electrode 40′).

Bonding layer 44 is disposed on a first surface of substrate 42. Because this surface will face shaft 18, it may be referred to as the back or reverse surface of substrate 42. As explained in greater detail below, bonding layer 44 is typically made of a material that can be reflow-bonded to shaft 18. Such materials, often referred to as “melt-processable materials,” will be familiar to those of ordinary skill in the art and include, by way of example only, polyether block amide (e.g., various grades of Pebax® (Arkema S. A., France)) and polyurethane.

Conductive layer 46 is disposed on a second surface of substrate 42 opposite bonding layer 44. Because this surface faces away from shaft 18, it may be referred to as the front surface of substrate 42. Conductive layer 46 likewise includes an exposed surface 48 and a reverse surface 50 (visible in FIG. 5B); reverse surface 50 of conductive layer 46 is generally adjacent the second (front) surface of substrate 42. Those of ordinary skill in the art will appreciate that a wide variety of electrically-conductive materials may be employed in the construction of conductive layer 46; exemplary materials include, without limitation, platinum, iridium, copper, gold, nickel, palladium, and combinations thereof.

To conductively couple electrode 40 (e.g., flexible ring electrode 40′) to a conductor extending along shaft 18 as discussed in greater detail below, a via 52 (visible in FIG. 5B) is provided through bonding layer 44 and substrate 42. Via 52 thus exposes a portion of reverse surface 50 of conductive layer 46.

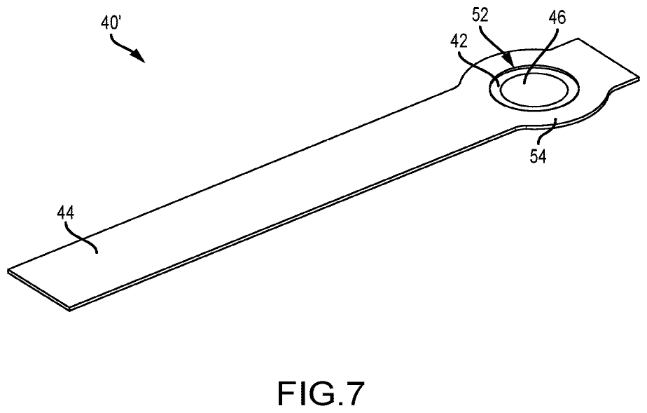

FIG. 7 illustrates that electrode 40 (e.g., flexible ring electrode 40′) is generally rectangular in plan shape, and that it assumes the ring configuration when wrapped around shaft 18 (or an analogous cylindrical core) as discussed below. To increase the amount of bonding surface area available, electrode 40 (e.g., flexible ring electrode 40′) may include a flared portion 54 proximate via 52 (e.g., to at least partially restore the material of bonding layer 44 that is removed to create via 52).

FIGS. 8A and 8B illustrate attachment of electrode 40 (e.g., flexible ring electrode 40′) to shaft 18. As seen in FIG. 8A, shaft 18 includes a conductive contact on an exterior surface thereof. The conductive contact may be a conductive (e.g., platinum) pill 56. In addition to being conductively coupled to electrode 40 (e.g., flexible ring electrode 40′) as described herein, conductive pill 56 may also be coupled to an elongate conductor that extends proximally through shaft 18 for interconnection to diagnostic and/or therapeutic equipment (e.g., an electroanatomical mapping system, such as Abbott Laboratories' EnSite Precision™M Cardiac Mapping System) at the proximal end of introducer 12 in a manner that will be well-understood by persons of ordinary skill in the art (and which will be discussed in greater detail below).

Alternatively, conductive pill 56 may be omitted, and the conductive contact may be a segment of the aforementioned elongate conductor that protrudes through the wall of shaft 12 (that is, electrode 40 (e.g., flexible ring electrode 40′) may be directly conductively coupled to the elongate conductor).

In any event, the conductive contact (e.g., conductive pill 56 or an analogous segment of an elongate conductor) may be part of a flexible circuit (e.g., a flexible electronic circuit) that is embedded in the wall of shaft 18 (e.g., during reflow processing of shaft 18 prior to attachment of electrodes 40 (e.g., flexible ring electrodes 40′)). The flexible circuit may be arranged generally parallel to the longitudinal axis of shaft 18.

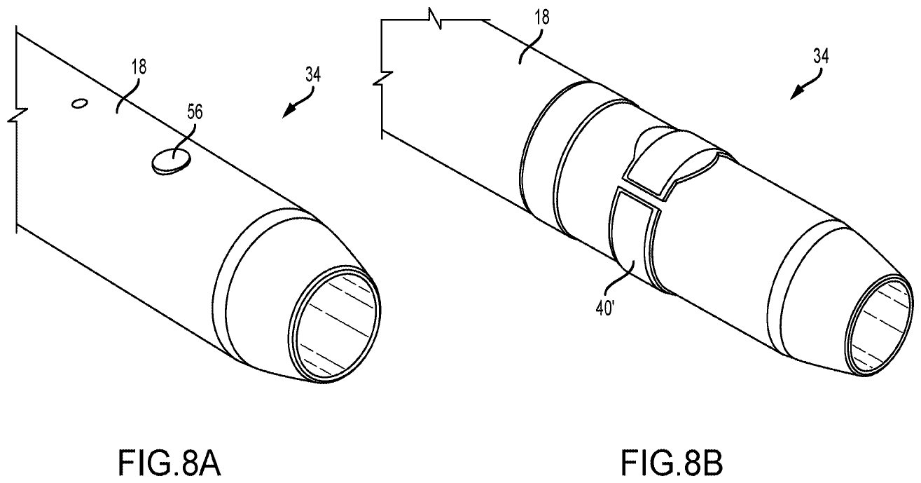

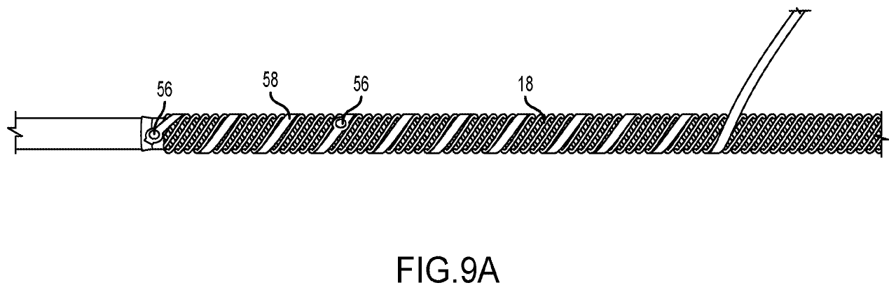

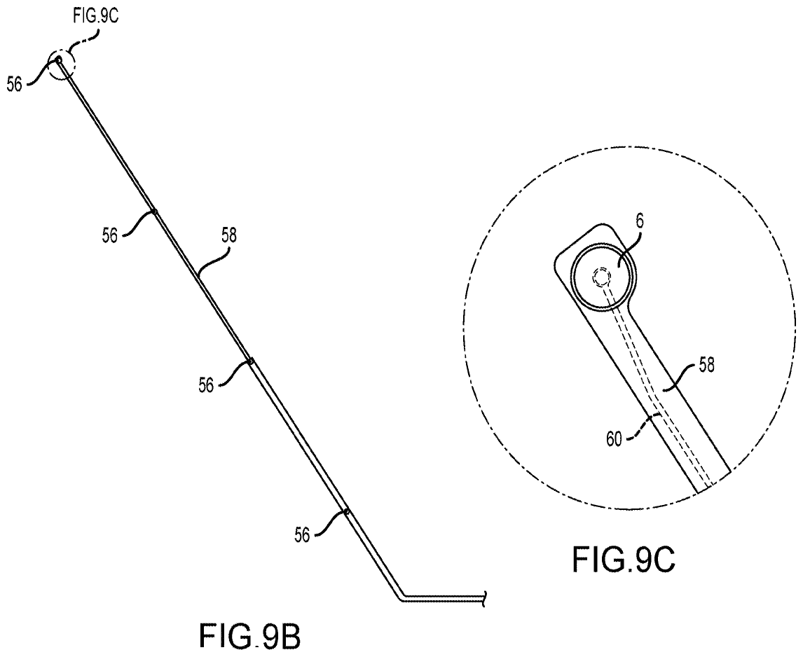

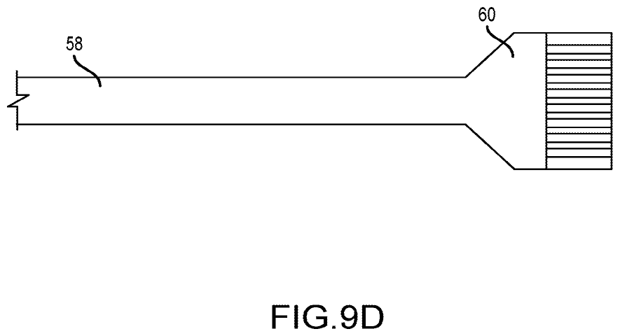

An exemplary flexible circuit 58 to which electrodes 40 (e.g., flexible ring electrodes 40′) may be conductively coupled is illustrated in FIGS. 9A-9C. As shown in FIGS. 9A-9C, flexible circuit 58 is wound in a spiral about shaft 18. Flexible circuit 58 may be wound at any angle relative to the longitudinal axis of shaft 18, may have any pitch (that is, spacing between turns), and may make any number of turns about shaft 18 along the length thereof without departing from the scope of the present disclosure.

FIG. 9B illustrates flexible circuit 58 in plan view (e.g., prior to being wrapped around shaft 18 during manufacture thereof). As shown in FIG. 9B, flexible circuit 58 includes four conductive pills 56 in its distal region; each conductive pill 56 can serve as a connection point for a corresponding electrode 40 (e.g., flexible ring electrode 40′) within distal region 34 of shaft 18. Of course, the number and spacing of conductive pills 56 shown in FIG. 9B are merely exemplary, and those of ordinary skill in the art will appreciate that they can be varied (e.g., to accommodate more or fewer electrodes 40 (e.g., flexible ring electrodes 40′) within distal region 34, to accommodate different spacings of electrodes 40 (e.g., flexible ring electrodes 40′) within distal region 34, and/or to accommodate different lengths of distal region 34′) without departing from the scope of the disclosure.

As shown in FIG. 9C, each conductive pill 56 can also be coupled to a corresponding conductive trace 60. Conductive traces 60 can run along the length of flexible circuit 58 to a proximal connector, such as a zero insertion force (ZIF) connector 62 as shown in FIG. 9D. Other connectors are, however, contemplated.

The substrate of flexible circuit 58 may be made of a polyimide, polyurethane, nylon, or the like.

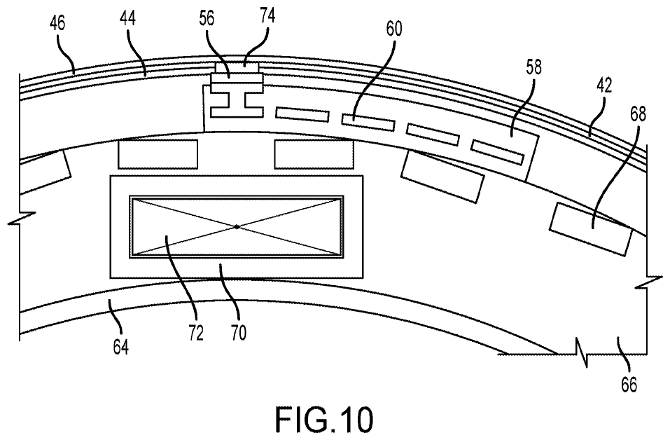

FIG. 10 is a close-up view of a portion of a transverse cross-section of shaft 18, flexible circuit 58 (e.g., spirally-wound flexible circuit), and electrode 40 (e.g., flexible ring electrode 40′). As mentioned above, the ordinarily-skilled artisan will appreciate various aspects of the construction of shaft 18. In this regard, inner liner 64, outer layer 66, braid layer 68, pull wire lumen 70, and pull wire 72 will be familiar to those of ordinary skill in the art and need not be further described herein.

Flexible circuit 58, including conductive traces 60, is wound spirally about shaft 18 radially outward of braid layer 68. Also visible in FIG. 10 are substrate 42, conductive layer 46, and bonding layer 44 of a representative electrode 40 (e.g., flexible ring electrode 40′), as well as conductive pill 56 and solder 74 to secure electrode 38 (e.g., flexible ring electrode 40′) to flexible circuit 58 (e.g., spirally-wound flexible circuit) as discussed above.

The assembly shown in FIG. 10 may be manufactured by reflow-bonding inner liner 64, outer layer 66, braid layer 68, pull wire lumen 70, pull wire 72, and flexible circuit 58 (e.g., spirally-wound flexible circuit as shown in FIG. 9A) in a first reflow-bonding step. The proximal end of flexible circuit 58 may be encased in a thermal barrier material during this first reflow bonding step to ensure that it remains free (e.g., not bonded to outer layer 66) for subsequent electrical connection (e.g., to ZIF connector 62).

In subsequent steps, one or more electrodes 40 (e.g., flexible ring electrodes 40′) may be bonded to corresponding conducive pill(s) 56 on flexible circuit 58 (e.g., spirally-wound flexible circuit) as described above. For instance, each electrode 40 (e.g., flexible ring electrode 40′) may be secured to a respective conductive pill 56 on flexible circuit 58 (e.g., spirally-wound flexible circuit) via soldering. As shown in FIG. 10, for example, a quantity of solder 74 may be applied to conductive pill 56 or, alternatively, to the exposed portion of reverse surface 50 of conductive layer 46. To help electrode 40 (e.g., flexible ring electrode 40′) lay flush on shaft 18, the thickness of the solder 64 so applied should approximate the combined thickness of substrate 42 and bonding layer 44.

Once electrode 40 (e.g., flexible ring electrode 40′) is positioned such that the exposed portion of reverse surface 50 of conductive layer 46 is in contact with conductive pill 56 via solder 74, solder 74 can be heated to create an electrically-conductive bond between the exposed portion of reverse surface 50 of conductive layer 46 and conductive pill 56. Thereafter, electrode 40 (e.g., flexible ring electrode 40′) can be wrapped around the perimeter of shaft 18 to form a ring electrode as shown in FIG. 8B. Bonding layer 44 can then be bonded to shaft 18, for example via a second reflow-bonding step as mentioned above.

As will be familiar to those of ordinary skill in the art, a layer of heat shrink material, such as a fluoropolymer or polyolefin material (e.g., polytetrafluoroethylene (PTFE) or fluorinated ethylene-propylene copolymer (FEP)), may be placed about the outer periphery of the assembly prior to the first and/or second reflow-bonding steps described above. As those of ordinary skill in the art will recognize, during reflow processing, energy (e.g., radiofrequency energy or thermal energy) is applied, for example to the outer surface of the assembly, to heat it to a point above the melting temperatures of the materials to be bonded (e.g., bonding layer 44 and outer layer 66, in the case of reflow bonding electrode 40 (e.g., flexible ring electrode 40′) to shaft 18). Any heat shrink layer should have a higher melting or softening temperature such that, during the reflow process, it will contract while retaining its tubular shape. This combination of applied energy and the pressure exerted by the heat shrink layer will force the melted materials (e.g., bonding layer 44 and outer layer 66) to flow and redistribute and, once cooled, the layers will be bonded to each other. The heat shrink layer can then be removed.

The foregoing method of manufacture offers various advantages over extant methods. For instance, because it bonds without swaging, it permits the use of non-circular (e.g., partial ring and/or ring segment) and non-metallic sensors. As another advantage, the reflow bonding process offers additional sealing against fluid ingress around flexible ring electrodes 40′. It also reduces cost and complexity of manufacture.

It should be understood, however, that additional and alternative methods of securing flexible ring electrode 40′ to conductive pill 56, such as swaging, resistance welding, laser welding, and the like, are also contemplated as within the scope of the present disclosure.

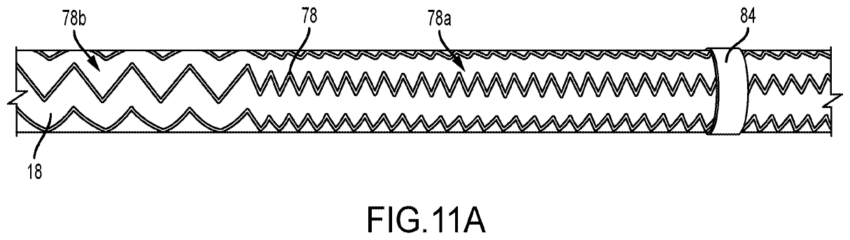

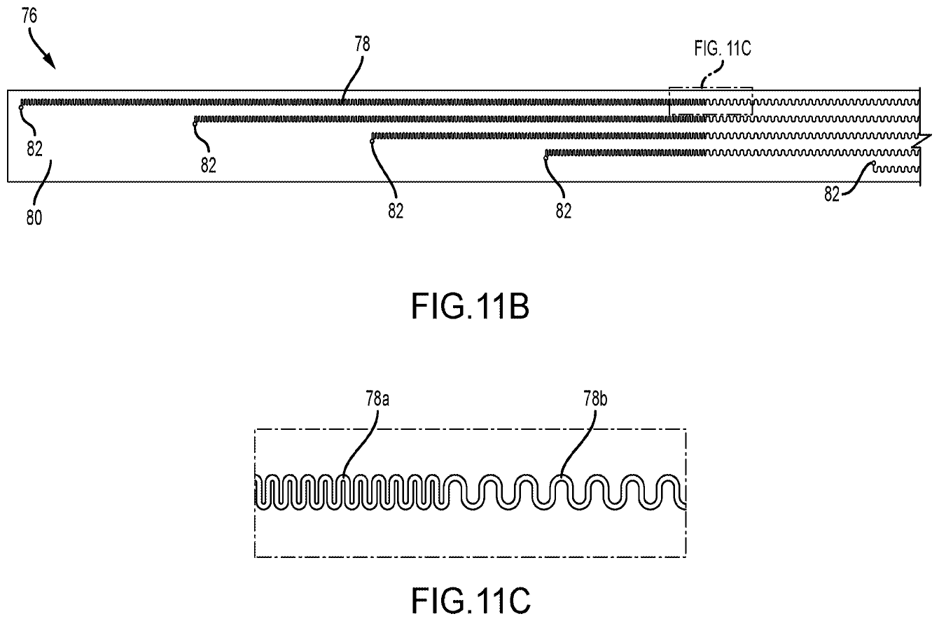

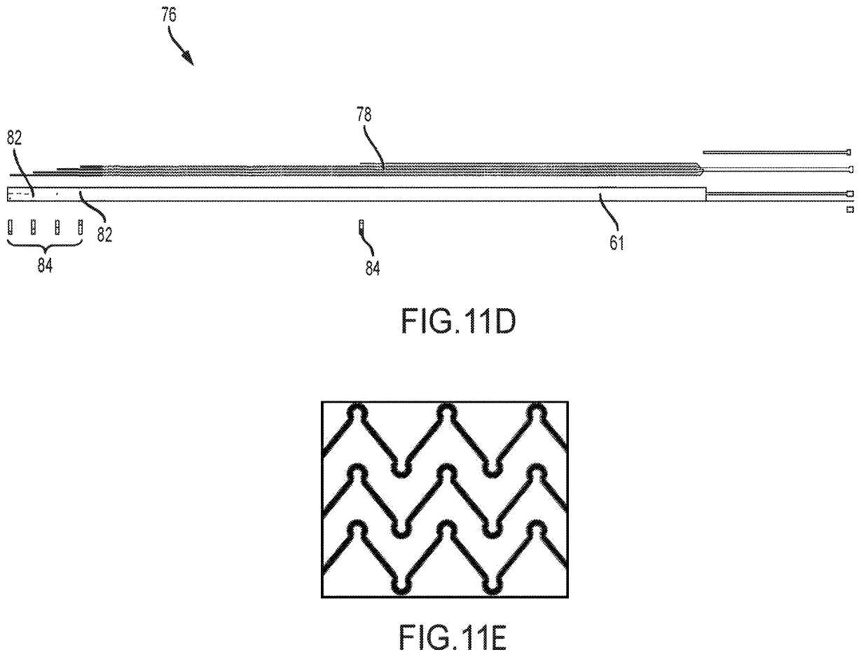

FIGS. 11A-11D depict alternative constructions of shaft 18 of introducer 12 incorporating various configurations of a flexible circuit 76. Although it is contemplated that the conductive traces of flexible circuit 76 may run in a substantially straight line along the length of flexible circuit 76 to a proximal connector (e.g., ZIF connector 59 as described above), other configurations are contemplated.

For instance, and as illustrated in FIGS. 11A-11C, conductive traces 78 of flexible circuit 76 may run in an undulating manner. As shown in FIG. 11A, undulating conductive traces 78 may run in a sawtooth (or zig-zag) manner along the length of flexible circuit 76 to a proximal connector; FIGS. 11B and 11C illustrate that undulating conductive traces 78 may instead run in a serpentine manner along the length of flexible circuit 76.

As further illustrated in FIGS. 11A and 11C in particular, the pitch of undulating (e.g., sawtooth/zig-zag or serpentine) conductive traces 78 can vary and can, for example, have a relatively narrower pitch 78a more distally and a relatively wider pitch 78b more proximally.

Still further, the undulation of conductive traces 78 may be interrupted and replaced with one or more segments having a different pattern at one or more locations along the length of flexible circuit 76. For instance, it may be desirable for conductive traces 78 to be generally straight, rather than undulating, in the vicinity of electrodes (e.g., electrodes 84 discussed below). To be clear, however, the term “generally straight” is not intended to mean, and should not be construed to require, that conductive traces 78 run parallel to the longitudinal axis of flexible circuit 76. Indeed, conductive traces 78 may run at an angle to the longitudinal axis of flexible circuit 76 and still be regarded as “generally straight” within the meaning of the instant disclosure.

FIG. 11E depicts an additional exemplary undulation pattern where the point at which the undulation reverses direction includes a looped portion. This pattern may be used along some or all of the length of conductive traces 78 and may advantageously facilitate strain relief within conductive traces 78 as shaft 18 of introducer 12 is maneuvered through the vasculature.

FIG. 11D is an exploded view of flexible circuit 76. As shown in FIG. 11D, undulating conductive traces 78 may be on a first side of a substrate 80, and a plurality of vias 82 may extend between the first side of substrate 80 and the second side of substrate 80, generally at the distal termination of undulating conductive traces 78. Electrodes 84, on the second side of substrate 80, may be respectively connected to conductive traces 78 through vias 82 in a manner familiar to those of ordinary skill in the art. This yields a conductor-substrate-conductor structure for flexible circuit 76.

Substrate 80 of flexible circuit 76 may be made of a polyether block amide (e.g., various grades of Pebax® (Arkema S. A., France)), nylon, polyurethane (e.g., thermoplastic polyurethane (TPU)), other thermoplastic elastomers (e.g., Santoprene™ (Celanese Corporation; Irving, TX)), and combinations thereof (e.g., different materials laminated together to collectively form substrate 80).

Thus, one possible structure for flexible circuit 76 is copper-PEBAX®-copper (e.g., copper undulating conductive traces 78-PEBAX® substrate 80-copper electrode(s) 84).

In another aspect of the disclosure, substrate 80 of flexible circuit 76 may be made of multiple layers of TPU, and the different layers may have different melting points. For instance, a TPU layer of substrate 80 that will abut the catheter shaft assembly (described below) may have a lower melting point than the TPU used in the remainder of the thickness of substrate 80. Thus, during reflow bonding of flexible circuit 76 to the catheter shaft assembly, the temperature can be made high enough to allow the TPU layer of substrate 80 that abuts the catheter shaft assembly to melt, facilitating bonding of flexible circuit 76 to the catheter shaft assembly, but low enough that the TPU used in the remainder of substrate 80 does not melt, helping preserve dimensional stability of conductive traces 78.

In another alternative, 80 of flexible circuit 76 may be made of a layer of a polyether block amide, with a relatively lower melting temperature, and a layer of TPU, with a relatively higher melting temperature, to achieve a similar objective as the TPU-TPU substrate described above.

Those of ordinary skill in the art will also appreciate additional materials that may be used, singly or in combination, in the construction of substrate 80.

Further, it is also contemplated that stretchable conductors may be used to form electrodes 84. Suitable stretchable conductors include, without limitation, single-and multi-walled carbon nanotube structures, copolymer elastomers, metal films (e.g., gold and/or copper in or on a polydimethylsiloxane substrate), metal coatings, metal flake conductive polymers (e.g., silver flakes in a polyurethane matrix), ion implanted materials, nanoparticle impregnated materials (e.g., copper and/or silver in a polyimide matrix), and nanoribbon/nanowire materials.

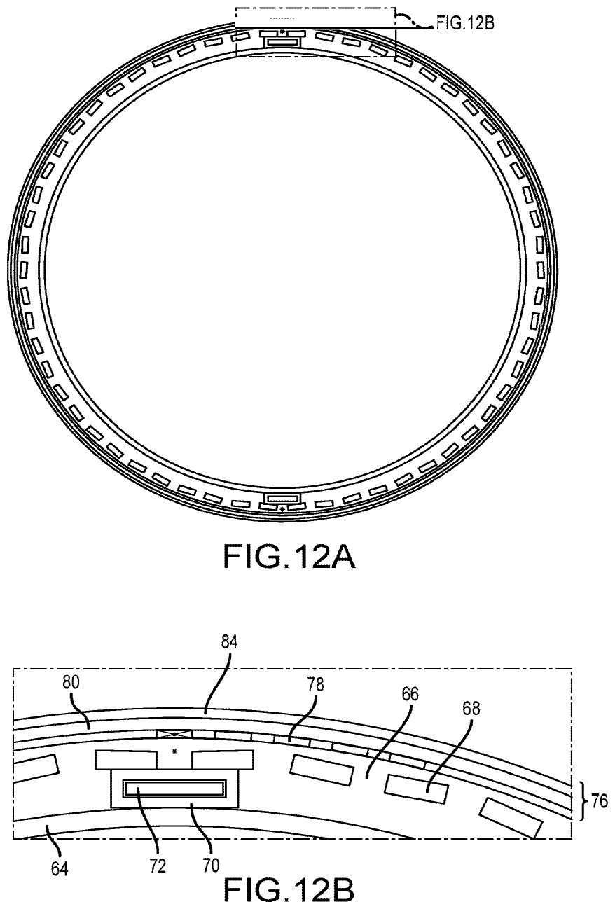

FIG. 12A depicts a transverse cross-section of shaft 18 including flexible circuit 76 with undulating conductive traces 76. FIG. 12B is a close-up view of box 12B in FIG. 12A illustrating the various components of shaft 18 according to aspects described herein. Similar to the discussion of FIG. 10 above, inner liner 64, outer layer 66, braid layer 68, pull wire lumen 70, and pull wire 72 will be familiar to those of ordinary skill in the art and need not be further described herein.

Also shown in FIG. 12B are undulating conductive traces 78 on flexible circuit 76. Substrate 80 and a representative electrode 84 are also visible in FIG. 12B.

The assembly shown in FIGS. 12A, 12B may be manufactured by first bonding inner liner 64, outer layer 66, braid layer 68, pull wire lumen 70, and pull wire 72 together via a first reflow-bonding step in a manner that will be familiar to the ordinarily-skilled artisan to thus form a catheter shaft assembly. Flexible circuit 76 with undulating conductive traces 78 may then be applied over the catheter shaft assembly before a second reflow-bonding step is carried out to bond flexible circuit 76 to the catheter shaft assembly. Similar to the discussion of flexible circuit 58 above, the proximal end of flexible circuit 76 may be wrapped in a thermal barrier material during this second reflow-bonding step to ensure that it remains free (e.g., not bonded to the catheter shaft assembly) for subsequent electrical connection (e.g., to ZIF connector 62).

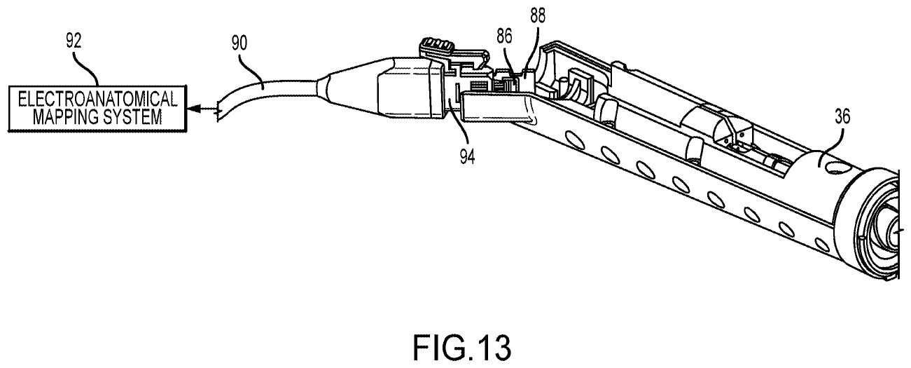

FIG. 13 depicts representative proximal electrical connections for introducer 12. As mentioned above, the proximal end of flexible circuit 58 or 76 can include a ZIF connector 62 for coupling to a corresponding ZIF receptacle 86 on a connector card 88. Connector card 88 can protrude proximally from handle 38 to facilitate connection to cable 90 leading to an electroanatomical mapping system 92. As with the connection of flexible circuit 58 or 76 to connector card 88, the connection between connector card 88 and cable 90 can also utilize a ZIF connector 94.

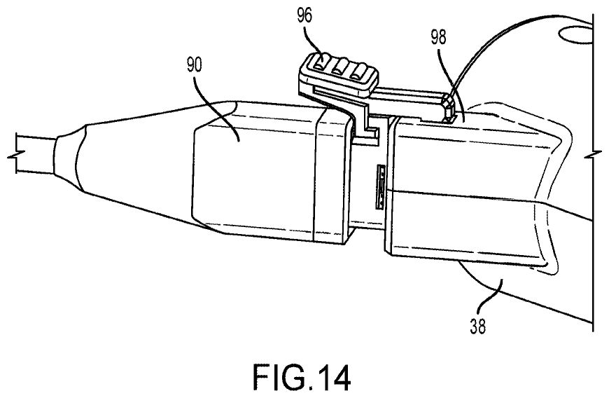

As shown in FIG. 14, cable 90 can further include a clip 96 to mate with a complementary component 98 on handle 38 (e.g., a tab-and-slot arrangement). This minimizes the risk that introducer 12 will be inadvertently disconnected from electroanatomical mapping system 92 during the course of a procedure.

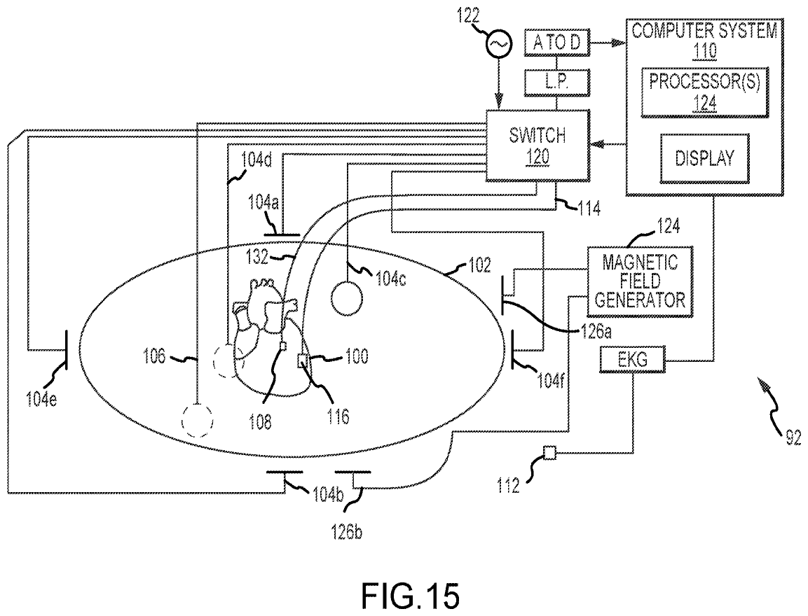

FIG. 15 shows a schematic diagram of an exemplary electroanatomical mapping system 92 as may be utilized in connection with the present teachings for conducting various diagnostic and therapeutic procedures, such as a cardiac electrophysiology study, by navigating a catheter (such as introducer 12 and/or other electrophysiology catheters) through a patient's vasculature. As those of ordinary skill in the art will recognize, system 92 can be used, for example, to create an anatomical model of the patient's heart 100 using one or more electrodes (e.g., electrodes 40 on introducer 12, which may include flexible ring electrodes 40′ and/or electrodes 84 as described above). System 92 can also be used to measure electrophysiology data at a plurality of points along a cardiac surface and store the measured data in association with location information for each measurement point at which the electrophysiology data was measured, for example to create a diagnostic data map of the patient's heart 100. System 92 can also be used to create three-dimensional visualizations of medical devices (e.g., introducer 12 as described above) within the patient's vasculature.

As one of ordinary skill in the art will recognize, system 92 determines the location, and in some aspects the orientation, of objects, typically within a three-dimensional space, and expresses those locations as position information determined relative to at least one reference.

This is referred to herein as “localization.”

For simplicity of illustration, the patient 102 is depicted schematically as an oval. In FIG. 15, three sets of surface electrodes (e.g., patch electrodes) 104a-104f are shown applied to a surface of the patient 102, pairwise defining three generally orthogonal axes, referred to herein as an x-axis, a y-axis, and a z-axis. Alternatively, the electrodes could be positioned in other arrangements, for example multiple electrodes on a particular body surface. As a further alternative, the electrodes do not need to be on the body surface but could be positioned internally to the body.

In FIG. 15, the x-axis surface electrodes are applied to the patient along a first axis, such as on the lateral sides of the thorax region of the patient (e.g., applied to the patient's skin underneath each arm) and may be referred to as the Left and Right electrodes. The y-axis electrodes are applied to the patient along a second axis generally orthogonal to the x-axis, along the sternum and spine of the patient in the thorax region, and may be referred to as the Chest and Back electrodes. The z-axis electrodes are applied along a third axis generally orthogonal to both the x-axis and the y-axis, such as along the inner thigh and neck regions of the patient, and may be referred to as the Left Leg and Neck electrodes. The heart 100 lies between these pairs of surface electrodes.

Each surface electrode 104a-104f can measure multiple signals. For example, each surface electrode can measure three resistance (impedance) signals and three reactance signals. These signals can, in turn, be grouped into three resistance/reactance signal pairs. One resistance/reactance signal pair can reflect driven values, while the other two resistance/reactance signal pairs can reflect non-drive values (e.g., measurements of the electric field generated by other driven pairs in a manner similar to that described below for electrodes 40).

An additional surface reference electrode (e.g., a “belly patch”) 106 provides a reference and/or ground electrode for the system 92. The belly patch electrode 106 may be an alternative to a fixed intra-cardiac electrode 108, described in further detail below. Alternatively, where system 92 is capable of magnetic field-based localization instead of or in addition to impedance-based localization, the surface electrode 106 can alternatively or additionally include a magnetic patient reference sensor-anterior (“PRS-A”) positioned on the patient's chest.

It should be appreciated that patient 102 may also have most or all of the conventional electrocardiogram (“ECG” or “EKG”) system leads in place. A standard set of 12 ECG leads may be utilized for sensing electrocardiograms on the patient's heart 100. This ECG information is available to system 92 (e.g., it can be provided as input to computer system 110). Insofar as ECG leads are well understood, and for the sake of clarity in the figures, only a single lead 112 and its connection to computer system 110 is illustrated in FIG. 15.

A representative catheter 114 (e.g., introducer 12) having at least one electrode 116 (e.g., electrodes 40) is also shown in FIG. 15. This representative catheter electrode 116 is sometimes referred to as a “roving electrode,” “moving electrode,” or “measurement electrode.” Multiple electrodes 116 on catheter 114, or on multiple such catheters, can be used.

As will be apparent from the foregoing description, catheter 114 can be used to simultaneously collect a plurality of electrophysiology data points. Each such electrophysiology data point includes both localization information (e.g., position of a unipole; position and orientation of a selected bipole) and corresponding electrogram signals (e.g., unipolar, bipolar, and/or omnipolar electrograms).

Catheter 114 (or multiple such catheters) are typically introduced into the heart and/or vasculature of the patient via one or more introducers (e.g., introducer 12) and using familiar procedures (e.g., the Seldinger technique of FIG. 1). Indeed, various approaches to introduce catheter 114 into a patient's heart, such as transseptal approaches, will be familiar to those of ordinary skill in the art, and therefore need not be further described herein.

Since each electrode 116 lies within the patient, location data may be collected simultaneously for each electrode 116 by system 92. Similarly, each electrode 116 can be used to gather electrophysiological data from the cardiac surface (e.g., endocardial electrograms). The ordinarily skilled artisan will be familiar with various modalities for the acquisition and processing of electrophysiology data points (including, for example, both contact and non-contact electrophysiological mapping), such that further discussion thereof is not necessary to the understanding of the techniques disclosed herein. Likewise, various techniques familiar in the art can be used to generate graphical representations of cardiac geometry and/or cardiac electrical activity from the plurality of electrophysiology data points. Moreover, insofar as the ordinarily skilled artisan will appreciate how to create electrophysiology maps from electrophysiology data points, the aspects thereof will only be described herein to the extent necessary to understand the present disclosure.

An optional fixed reference electrode 108 (e.g., attached to a wall of the heart 100) is shown on a second catheter 118. For calibration purposes, this electrode 108 may be stationary (e.g., attached to or near the wall of the heart) or disposed in a fixed spatial relationship with the roving electrodes (e.g., electrodes 116), and thus may be referred to as a “navigational reference” or “local reference.” The fixed reference electrode 108 may be used in addition or alternatively to the surface reference electrode 106 described above. In many instances, a coronary sinus electrode or other fixed electrode in the heart 100 can be used as a reference for measuring voltages and displacements; that is, as described below, fixed reference electrode 108 may define the origin of a coordinate system.

Each surface electrode is coupled to a multiplex switch 120, and the pairs of surface electrodes are selected by software running on computer system 110, which couples the surface electrodes to a signal generator 122. Alternately, switch 120 may be eliminated and multiple (e.g., three) instances of signal generator 122 may be provided, one for each measurement axis (that is, each surface electrode pairing).

Computer system 110 may comprise, for example, a conventional general-purpose computer, a special-purpose computer, a distributed computer, or any other type of computer. Computer system 110 may comprise one or more processors 124, such as a single central processing unit (“CPU”), or a plurality of processing units, commonly referred to as a parallel processing environment, which may execute instructions to practice the various aspects described herein.

Generally, three nominally orthogonal electric fields are generated by a series of driven and sensed electric dipoles (e.g., pairs of surface electrodes 104a-104f) in order to realize catheter navigation in a biological conductor. Alternatively, these orthogonal fields can be decomposed and any pairs of surface electrodes can be driven as dipoles to provide effective electrode triangulation. Likewise, the electrodes 104a-104f (or any number of electrodes) could be positioned in any other effective arrangement for driving a current to or sensing a current from an electrode in the heart. For example, multiple electrodes could be placed on the back, sides, and/or belly of patient 102. Additionally, such non-orthogonal methodologies add to the flexibility of the system. For any desired axis, the potentials measured across the roving electrodes resulting from a predetermined set of drive (source-sink) configurations may be combined algebraically to yield the same effective potential as would be obtained by simply driving a uniform current along the orthogonal axes.

Thus, any two of the surface electrodes 104a-104f may be selected as a dipole source and drain with respect to a ground reference, such as belly patch 106, while the unexcited electrodes measure voltage with respect to the ground reference. The roving electrodes 116 placed in the heart 100 are exposed to the field from navigational currents and are measured with respect to ground, such as belly patch 106. As previously noted, at least one electrode may be fixed to the interior surface of the heart to form a fixed reference electrode 108, which is also measured with respect to ground, such as belly patch 106, and which may be defined as the origin of the coordinate system relative to which system 92 measures positions. Data sets from each of the surface electrodes, the internal electrodes, and the virtual electrodes may all be used to determine the location of the roving electrodes 116 within heart 100.

The measured voltages may be used by system 92 to determine the location in three-dimensional space of the electrodes inside the heart, such as roving electrodes 116 relative to a reference location, such as reference electrode 108. That is, the voltages measured at reference electrode 108 may be used to define the origin of a coordinate system, while the voltages measured at roving electrodes 116 may be used to express the location of roving electrodes 116 relative to the origin. The coordinate system may be a three-dimensional (x, y, z) Cartesian coordinate system, although other coordinate systems, such as polar, spherical, and cylindrical coordinate systems, are contemplated.

As should be clear from the foregoing discussion, the data used to determine the location of the electrode(s) within the heart is measured while the surface electrode pairs impress an electric field on the heart. The electrode data may also be used to create a respiration compensation value used to improve the raw location data for the electrode locations as described, for example, in U.S. Pat. No. 7,263,397, which is hereby incorporated herein by reference in its entirety. The electrode data may also be used to compensate for changes in the impedance of the body of the patient as described, for example, in U.S. Pat. No. 7,885,707, which is also incorporated herein by reference in its entirety.

Therefore, in a representative system, system 92 first selects a set of surface electrodes and then drives them with current pulses. While the current pulses are being delivered, electrical activity, such as the voltages measured with at least one of the remaining surface electrodes and in vivo electrodes, is measured and stored. Compensation for artifacts, such as respiration and/or impedance shifting, may be performed as indicated above.

In aspects of the disclosure, system 92 can be a hybrid system that incorporates both impedance-based (e.g., as described above) and magnetic-based localization capabilities. Thus, for example, system 92 can also include a magnetic source 124, which is coupled to one or more magnetic field generators. In the interest of clarity, only two magnetic field generators 126a and 126b are depicted in FIG. 15, but it should be understood that additional magnetic field generators (e.g., a total of six magnetic field generators, defining three generally orthogonal axes analogous to those defined by patch electrodes 104a-104f) can be used without departing from the scope of the present teachings. Likewise, those of ordinary skill in the art will appreciate that, for purposes of localization within the magnetic fields so generated, catheter 114 (e.g., introducer 12) can include one or more magnetic localization sensors (e.g., coils).

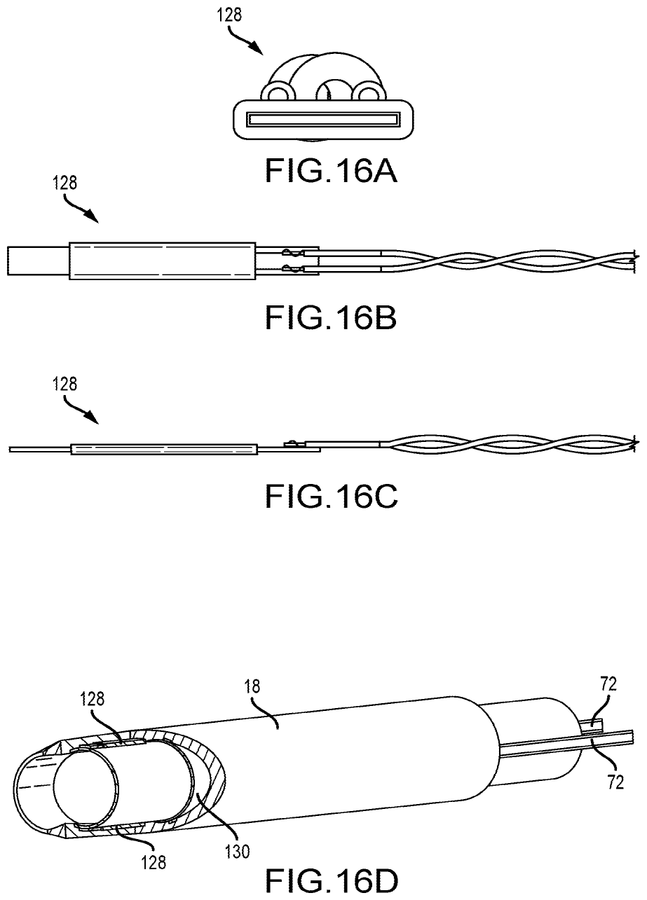

In this respect, FIGS. 16A through 16C are front, top, and side views, respectively, of a first representative magnetic localization sensor 128, referred to herein as a “flat magnetic localization sensor.” FIG. 16D illustrates the positioning of flat magnetic localization sensor 128 on shaft 18—specifically, two flat magnetic localization sensors 128 are positioned at about 180 degrees spacing around shaft 18 distal of a pull ring 130 to which pull wires 72 are secured.

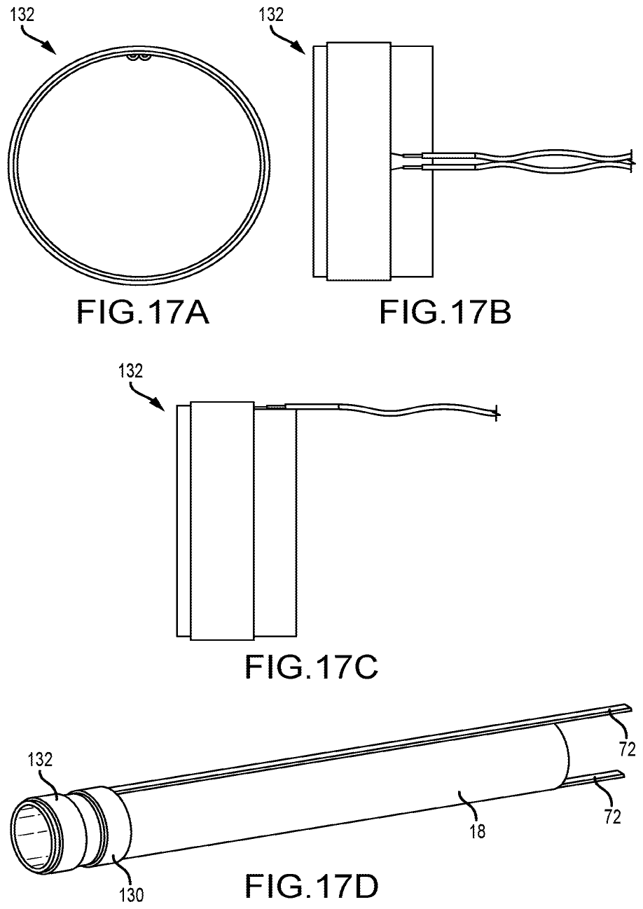

FIGS. 17A through 17C are front, top, and side views, respectively, of a second representative magnetic localization sensor 132, referred to herein as a “hollow” magnetic localization sensor. FIG. 17D illustrates the positioning of hollow magnetic localization sensor 132 on shaft 18—specifically, distal of pull ring 130 to which pull wires 72 are secured.

Further details of suitable magnetic localization sensors are provided in U.S. provisional Application No. 63/553,414 , which is hereby incorporated by reference as though fully set forth herein.

System 92 may be the EnSite™ X, EnSite™ Velocity™, or EnSite Precision™ electrophysiological mapping and visualization system of Abbott Laboratories. Other localization systems, however, may be used in connection with the present teachings, including for example the RHYTHMIA HDX™ mapping system of Boston Scientific Corporation (Marlborough, Massachusetts), the CARTO navigation and location system of Biosense Webster, Inc. (Irvine, California), the AURORA® system of Northern Digital Inc. (Waterloo, Ontario), Stereotaxis, Inc.'s NIOBE® Magnetic Navigation System (St. Louis, Missouri), as well as MediGuide™ Technology from Abbott Laboratories.

The localization and mapping systems described in the following patents (all of which are hereby incorporated by reference in their entireties) can also be used with the present invention: U.S. Pat. Nos. 6,990,370; 6,978,168; 6,947,785; 6,939,309; 6,728,562; 6,640,119; 5,983,126; and 5,697,377.

Although several configurations and methods of manufacture have been described above with a certain degree of particularity, those skilled in the art could make numerous alterations to the disclosed flexible electrodes, catheters, and methods of manufacture without departing from the spirit or scope of this invention.

For example, in addition to electrodes 40 within distal region 34, shaft 18 may also include one or more electrodes within its proximal region.

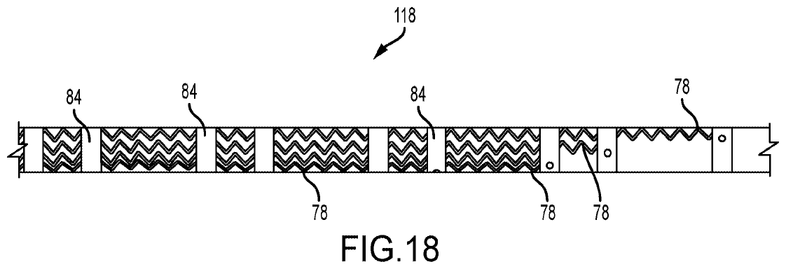

As another example, and as mentioned above, the teachings herein can be applied to any number of catheters or similar medical devices, including, but not limited to, steerable diagnostic and therapeutic catheters (e.g., electrophysiology mapping and/or ablation catheters), coronary sinus catheters, and the like. For instance, FIG. 18 depicts the use of flexible circuit 76 including a plurality of undulating conductive traces 78 respectively coupled to electrodes 84 in a coronary sinus catheter 118 (e.g., as shown schematically in FIG. 15) (for convenience, only the distal-most portion of coronary sinus catheter 118 is shown in FIG. 18).

Those of ordinary skill in the art will likewise recognize that a multi-electrode electrophysiology mapping catheter may be configured substantially similarly to coronary sinus catheter 118 depicted in FIG. 18.

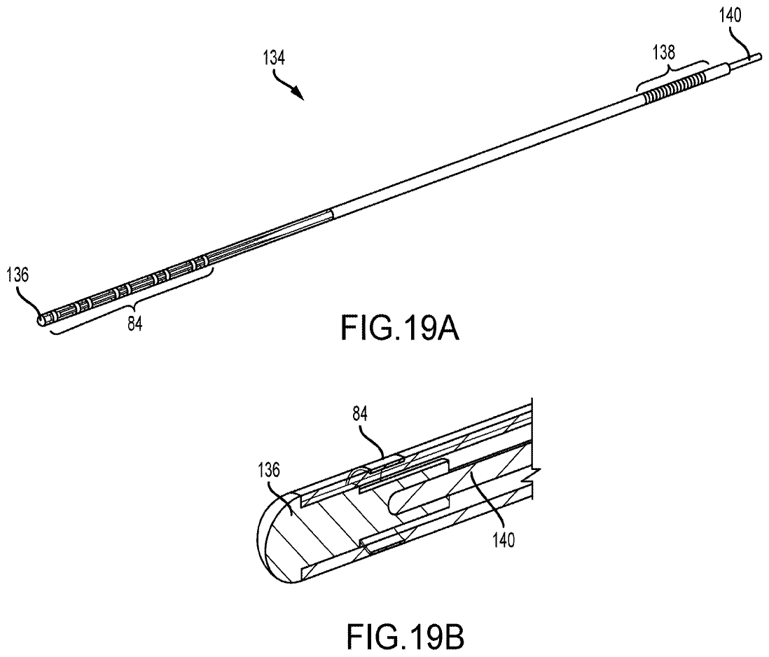

A configuration similar to that shown in FIG. 18 may also be applied to good advantage in a device for crossing the interatrial septum using radiofrequency energy (also referred to herein as “RF-based transseptal puncture apparatus”). An exemplary RF-based transseptal apparatus 134 is shown in FIGS. 19A and 19B. RF-based transseptal apparatus 134 can include a plurality of electrodes 84 on a flexible circuit 76 as described above, as well as a tip electrode 136, in a distal segment.

As described above, undulating conductive traces 78 connect electrodes 84 to respective proximal connector pads 138 in a proximal segment of RF-based transseptal apparatus 134. It is contemplated that the distal segment of RF-based transseptal apparatus 134 may be made of a softer material than the proximal segment of RF-based transseptal apparatus 134.

Tip electrode 136 is connected to a conductive core 140, also shown in cutaway in FIG. 19B. Conductive core 140 may be made of a malleable material, which allows a practitioner to plastically deform device 140 into a desired configuration. The malleable material may, for example, be stainless steel.

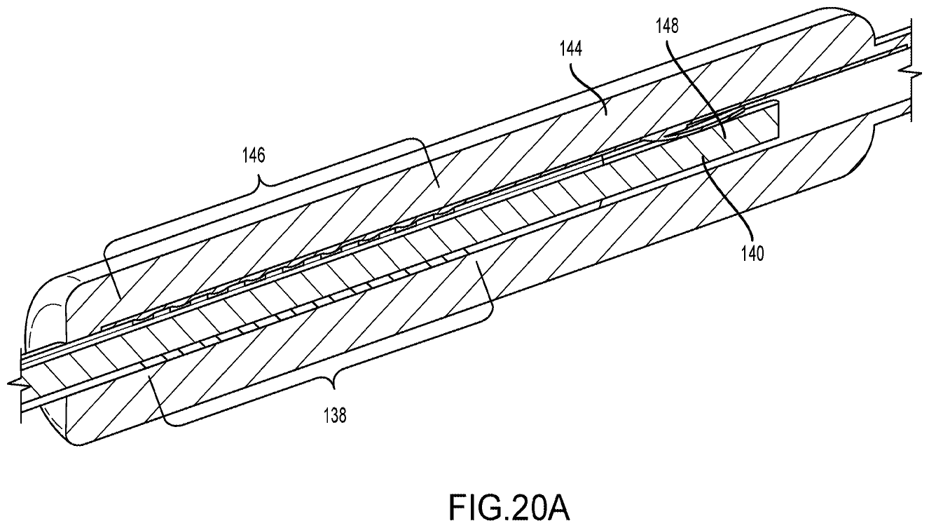

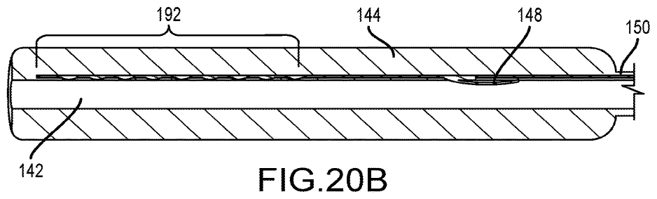

FIGS. 20A and 20B depict proximal electrical connections for RF-based transseptal apparatus 134 of FIGS. 19A and 19B. When the proximal end of RF-based transseptal apparatus 134 is inserted into cavity 142 of connector housing 144, proximal connector pads 138 conductively couple to corresponding electrical connectors 146. Similarly, conductive core 140 conductively couples to spring electrical connector 148. In turn, extension cable 150 connects, for example, to system 92.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

### NUMBERED CLAUSES OF THE INVENTION

1. A flexible electrode, comprising:
a flexible, electrically insulative substrate;
a bonding layer disposed on a first surface of the substrate;
a conductive layer disposed on a second surface of the substrate opposite the first surface of the substrate, the conductive layer having an exposed surface and a reverse surface, wherein the reverse surface is adjacent the second surface of the substrate; and
a via through the bonding layer and the substrate that exposes a portion of the reverse surface of the conductive layer.

2. The flexible electrode according to clause 1, wherein the flexible electrode has a rectangular plan shape that, when wrapped about a cylindrical core, defines a ring electrode, optionally, wherein the rectangular plan shape of the flexible electrode includes a flared portion proximate the via.

3. The flexible electrode according to any of clauses 1 or 2, wherein the substrate comprises polyimide.

4. The flexible electrode according to any of clauses 1 to 3, wherein the bonding layer comprises a melt-processable material, optionally, wherein the bonding layer comprises one or more of polyether block amide (PEBA) and polyurethane.

5. The flexible electrode according to any of clauses 1 to 4, wherein the conductive layer comprises one or more of platinum, iridium, copper, gold, nickel, and palladium.

6. A method of manufacturing a catheter, the method comprising:
forming a catheter shaft including a conductive contact adjacent an exterior surface thereof;
forming a flexible electrode comprising:
   a flexible, electrically insulative substrate;
   a bonding layer disposed on a first surface of the substrate;
   a conductive layer disposed on a second surface of the substrate opposite the first surface of the substrate, the conductive layer having an exposed surface and a reverse surface, wherein the reverse surface is adjacent the second surface of the substrate; and
   a via through the bonding layer and the substrate that exposes a portion of the reverse surface of the conductive layer;
securing the portion of the reverse surface of the conductive layer to the conductive contact;
wrapping the flexible electrode about the exterior surface of the catheter shaft to form a ring electrode; and
bonding the bonding layer to the catheter shaft.

7. The method according to clause 6, wherein securing the portion of the reverse surface of the conductive layer to the conductive contact comprises soldering the portion of the reverse surface of the conductive layer to the conductive contact.

8. The method according to any of clauses 6 or 7, wherein bonding the bonding layer to the catheter shaft comprises reflow bonding the bonding layer to the catheter shaft, optionally, wherein the method further comprises placing a heat shrink around the flexible electrode prior to reflow bonding the bonding layer to the catheter shaft.

9. The method according to any of clauses 6 to 8, wherein the conductive contact comprises a conductive pill adjacent the exterior surface of the catheter shaft.

10. The method according to any of clauses 6 to 8, wherein the conductive contact comprises a first segment of an elongate electrical conductor, wherein a second segment of the elongate electrical conductor extends through the catheter shaft.

11. A catheter comprising:
an elongate shaft; and
a ring electrode mounted to the elongate shaft, wherein the ring electrode comprises:
   a flexible, electrically insulative substrate;
   a bonding layer disposed on a first surface of the substrate;
   a conductive layer disposed on a second surface of the substrate opposite the first surface of the substrate, the conductive layer having an exposed surface and a reverse surface, wherein the reverse surface is adjacent the second surface of the substrate; and
   a via through the bonding layer and the substrate that exposes a portion of the reverse surface of the conductive layer.

12. The catheter according to clause 11, wherein the elongate shaft further comprises a conductive contact, and wherein the exposed portion of the reverse surface is bonded to the conductive contact, optionally, wherein the exposed portion of the reverse surface is soldered to the conductive contact.

13. The catheter according to clause 12, further comprising an elongate electrical conductor extending through the elongate shaft from the conductive contact, or, wherein the conductive contact comprises a segment of the elongate electrical connector.

14. The catheter according to any of clauses 11 to 13, wherein the bonding layer is reflow bonded to the elongate shaft.

15. The catheter according to any of clauses 11 to 14, wherein the ring electrode comprises a flared portion proximate the via.

1-20. (canceled)

21. A catheter comprising:
an elongate shaft defining a lumen;
a flexible circuit extending along a length of the elongate shaft, the flexible circuit including a substrate and an undulating conductive trace disposed on the substrate; and
an electrode conductively coupled to the undulating conductive trace.

22. The catheter according to claim 21, wherein the electrode is part of the flexible circuit.

23. The catheter according to claim 22, wherein the undulating conductive trace is disposed on a first surface of the substrate and the electrode is disposed on a second surface of the substrate, and wherein the flexible circuit further comprises a conductive via through the substrate to conductively couple the electrode to the undulating conductive trace.

24. The catheter according to claim 21, wherein the elongate shaft comprises a first material, the substrate comprises a second material, and the first material and the second material are compatible to be reflow bonded together.

25. The catheter according to claim 24, wherein the second material is selected from the group consisting of polyether block amide, nylon, polyurethane, thermoplastic elastomer, and combinations thereof.

26. The catheter according to claim 21, wherein the undulating conductive trace comprises a zig-zag pattern.

27. The catheter according to claim 21, wherein the undulating conductive trace comprises a serpentine pattern.

28. The catheter according to claim 21, wherein the undulating conductive trace comprises a variable pitch undulation pattern.

29. The catheter according to claim 28, wherein a pitch of the variable pitch undulation pattern is narrower within a distal portion of the flexible circuit than within a proximal portion of the flexible circuit.

30. The catheter according to claim 21, wherein the undulating conductive trace comprises an undulation pattern including an interruption.

31. The catheter according to claim 30, wherein the interruption comprises a straight segment.

32. The catheter according to claim 31, wherein the straight segment is proximate the electrode.

33. The catheter according to claim 21, wherein the undulating conductive trace comprises a plurality of loops where a direction of undulation reverses.

34. The catheter according to claim 21, wherein the electrode comprises a stretchable conductor.

35. The catheter according to claim 34, wherein the stretchable conductor is selected from the group consisting of a single-walled carbon nanotube structure; a multi-walled carbon nanotube structure; a copolymer elastomer; a metal film; a metal coating; a metal flake conductive polymer; an ion-implanted material; a nanoparticle impregnated material; a nanoribbon material; a nanowire material; and combinations thereof.

36. The catheter according to claim 21, further comprising a handle coupled to a proximal end of the elongate shaft.

37. The catheter according to claim 36, wherein the handle comprises a steering assembly and further comprising a pull wire extending along the length of the elongate shaft, a distal end of the pull wire being secured to a distal portion of the elongate shaft and a proximal end of the pull wire being secured to the steering assembly.

38. The catheter according to claim 21, further comprising an electrical connector coupled to a proximal end of the flexible circuit.

39. The catheter according to claim 38, wherein the electrical connector comprises a zero insertion force connector.

40. The catheter according to claim 21, wherein the undulating conductive trace comprises a plurality of undulating traces and the electrode comprises a plurality of electrodes, each electrode of the plurality of electrodes being conductively coupled to a respective undulating trace of the plurality of undulating traces, and wherein each undulating trace of the plurality of undulating traces has a distal termination point at a different position along a length of the flexible circuit.
